(19) United States
(12) Patent Application Publication
(10) Pub. No.: US 2014/0335578 A1
(43) Pub. Date: Nov. 13, 2014

(54) INTEGRATED BIODIESEL PROCESS

(71) Applicant: William Marsh Rice University, Houston, TX (US)

(72) Inventors: Ka-Yiu SAN, Houston, TX (US); Hui WU, Houston, TX (US)

(73) Assignee: William Marsh Rice University, Houston, TX (US)

(21) Appl. No.: 14/266,942

(22) Filed: May 1, 2014

Related U.S. Application Data
(60) Provisional application No. 61/820,929, filed on May 8, 2013.

(51) Int. Cl. C12P 7/64

(52) U.S. Cl. CPC C12P 7/649
USPC 435/134; 435/252.3

(57) ABSTRACT

Methods of using crude glycerol to make fatty acids are provided, as well as integrated methods of converting glycerol waste from biodiesel production into more biodiesel. Bacteria and other microbes engineered to produce free fatty acids from glycerol are also provided.

### PRIOR RELATED APPLICATIONS

This application claims priority to 61/820,929, titled INTEGRATED BIODIESEL PROCESS and filed May 8, 2013, and which is expressly incorporated by reference herein for all purposes.

### FEDERALLY SPONSORED RESEARCH STATEMENT

Not applicable.

### REFERENCE TO MICROFICHE APPENDIX

Not applicable.

### FIELD OF THE DISCLOSURE

Large quantities of glycerol are being produced in biodiesel manufacturing processes as waste. The current invention makes use of this glycerol to produce free fatty acids, which can be used to produce more biodiesel without producing added glycerol as a waste product.

### BACKGROUND OF THE DISCLOSURE

Biodiesel is an oil- or fat-based diesel fuel consisting of long-chain alkyl esters. The National Biodiesel Board also has a technical definition of biodiesel as a mono-alkyl ester. Biodiesel is typically made by chemically reacting lipids (e.g., vegetable oil or animal fat (tallow)) with an alcohol to produce fatty acid esters according to reaction (1), wherein triglycerides (1) are reacted with an alcohol such as ethanol (2) to give ethyl esters of fatty acids (3) and glycerol (4):

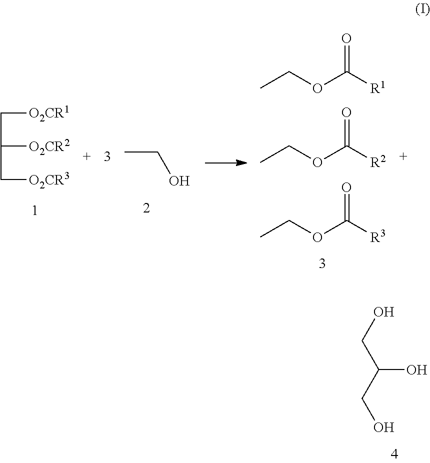

Biodiesel is used in standard diesel engines and is thus distinct from the vegetable and waste oils used to fuel converted diesel engines. It can be used alone, or blended with petrodiesel. Biodiesel can also be used as a low carbon alternative to heating oil. As such it has been an important component in global efforts to reduce petroleum consumption, and move towards sustainable energy production models.

A variety of oils and fats can be used to produce biodiesel. These include:
Virgin oil feedstock—rapeseed and soybean oils are most commonly used, soybean oil accounting for about half of U.S. production. It also can be obtained from Pongamia, field pennycress, jatropha and other crops such as mustard, jojoba, flax, sunflower, palm oil, coconut, hemp, and the like.
Waste vegetable oil (WVO).
Animal fats including tallow, lard, yellow grease, chicken fat, and the by-products of the production of omega-3 fatty acids from fish oil.
Algae, which can be grown using waste materials such as sewage and without displacing land currently used for food production.
Oil from halophytes, such as Salicornia bigelovii, which can be grown using saltwater in coastal areas where conventional crops cannot be grown, with yields equal to the yields of soybeans and other oilseeds grown using freshwater irrigation.
Sewage Sludge—The sewage-to-biofuel field is attracting interest from major companies like Waste Management and startups like InfoSpi, which are betting that renewable sewage biodiesel can become competitive with petroleum diesel on price.

As shown in reaction (I) above, glycerol is the major byproduct produced during the production of biodiesel from fat or oil. Indeed, about one pound glycerol is formed per every 10 pounds of biodiesel produced and global productions levels have long topped two million cubic meters of glycerol annually (FIG. 1) resulting in a 67% price decrease in US glycerol due to the market glut. Therefore, there is increasing interest in developing uses for the excess glycerol—now considered a waste product rather than a valuable chemical.

Crude glycerol, however, is not very pure, containing methanol, salts, soaps and water as the main contaminants. Concentration and presence of each contaminant will vary drastically from one industry to another, due to a variety of parameters, including oil source and reaction conditions. For instance, glycerol can vary from 92% to 65% in crude glycerol samples, and water can vary from 6-26%. The presence of such impurities in crude glycerol samples can negatively impact any conversion process of this waste product.

Various studies have been reported to convert waste glycerol into more valuable products such as ethanol, succinate and polyhydroxybutyrate (PHB), omega-3 polyunsaturated fatty acids, 1,3 propanediol (PDO). However, such methods usually require transport of crude glycerol to another facility where the conversion can take place, thus increasing costs and reducing efficiencies (see FIG. 2A). Furthermore, the impurities present in crude glycerol have complicated such methods.

Thus, what is needed in the art is a method of using the vast amounts of crude glycerol being produced annually, preferably a cost efficient method that can be integrated with existing biodiesel production.

### SUMMARY OF THE DISCLOSURE

Our approach for converting crude glycerol waste product into a valuable commodity is to biologically convert the glycerol into free fatty acids that can be used directly as a feedstock to produce more biodiesel (FIG. 2B). The appeal of this scheme is that biodiesel is the only major product, making the integrated biodiesel production process more efficient. Of course, the free fatty acids can also be sold to the market, or used as feedstocks for other chemical synthesis reactions, as desired.

The steps involved in implementing the process are:

1) Engineering a microbial strain to efficiently convert glycerol to free fatty acids.

2) Assembling a bioreactor to grow the engineered strain to process the glycerol from a biodiesel plant. Preferably, the reactor is at the same facility as the biodiesel production facility, thus glycerol is separated from the biodiesel and/or fatty acids, and fed directly to the bioreactor.

3) Free fatty acids can be removed from the bioreactor, and fed back into the system for biodiesel production, or a separate reactor can convert the fatty acids to biodiesel, which is then combined with biodiesel from the remaining part of the facility. Alternatively, the fatty acids can be sold as is, or used for the synthesis of other derivatives, and the like.

The advantages of using the current system of producing free fatty acid from glycerol include:
It recycles the glycerol byproduct to form more biodiesel, which is a highly desirable product.
Only a single product is produced, and therefore the method allows implementation of a more efficient business model.

In various embodiments, the invention includes a method of biodiesel production comprising: converting a biologically produced fat or oil to biodiesel and glycerol; separating said biodiesel and said glycerol; converting the glycerol to free fatty acids in a reactor with a recombinant microbe; separating said fatty acids from said microbe; converting the separated free fatty acids to biodiesel. The invention can also be used to make fatty acids, which can be used for other purposes.

The method includes the use of a recombinant microbe that overexpresses a TE. The microbe can also comprise one or more of the overexpression or reductions listed in Table 7 or FIG. 6, or any bacteria of Tables 2-6 and FIG. 6, or any bacteria, yeast or algae herein described.

Preferably, the microbe makes at least 0.25 g/L of free fatty acid, but greater amounts are possible including >0.5, 0.75, 1, 2, 3 or even >4 g/L of free fatty acid.

Many microbes do not make significant amounts of free fatty acids, but can be made to do so by adding a gene coding for an acyl-ACP thioesterase (called a “TE” gene herein). Acyl-acyl carrier protein (ACP) thioesterase is an enzyme that terminates the intraplastidial fatty acid synthesis by hydrolyzing the acyl-ACP intermediates and releasing free fatty acids to be incorporated into glycerolipids. These enzymes are classified in two families, FatA and FatB, which differ in amino acid sequence and substrate specificity. Generally speaking, the N terminal (aa 1-98) of any acyl-ACP thioesterase controls the substrate specificity of the enzyme, and it is known how to change substrate specificity by swapping amino terminal domains.

Many acyl-ACP thioesterase proteins are known and can be added to bacteria for use in the invention (e.g., CAA52070, YP_{—}003274948, ACY23055, AAB71729, BAB33929, to name a few of the thousands of such proteins available), although we have used plasmids encoded plant genes herein. Such genes can be added by plasmid or other vector, or can be cloned directly into the genome. In certain species it may also be possible to genetically engineer the endogenous protein to be overexpressed by changing the regulatory sequences or removing repressors. However, overexpressing the gene by inclusion on selectable plasmids that exist in hundreds of copies in the cell may be preferred due to its simplicity, although permanent modifications to the genome may be preferred in the long term for stability reasons.

Other fatty acyl ACP thioesterases include Umbellularia californica (GenBank #AAC49001), Cinnamomum camphora (GenBank #Q39473), Umbellularia californica (GenBank #Q41635), Myristica fragrans (GenBank #AAB71729), Myristica fragrans (GenBank #AAB71730), Elaeis guineensis (GenBank #ABD83939), Elaeis guineensis (GenBank #AAD42220), Populus tomentosa (GenBank #ABC47311), Arabidopsis thaliana (GenBank #NP-172327), Arabidopsis thaliana (GenBank #CAA85387), Arabidopsis thaliana (GenBank #CAA85388), Gossypium hirsutum (GenBank #Q9SQI3), Cuphea lanceolata (GenBank #CAA54060), Cuphea hookeriana (GenBank #AAC72882), Cuphea calophylla subsp. mesostemon (GenBank #ABB71581), Cuphea lanceolata (GenBank #CAC19933), Elaeis guineensis (GenBank #AAL15645), Cuphea hookeriana (GenBank #Q39513), Gossypium hirsutum (GenBank #AAD01982), Vitis vinifera (GenBank #CAN81819), Garcinia mangostana (GenBank #AAB51525), Brassica juncea (GenBank #ABI18986), Madhuca longifolia (GenBank #AAX51637), Brassica napus (GenBank #ABH11710), Oryza sativa (indica cultivar-group) (GenBank #EAY86877), Oryza sativa (japonica cultivar-group) (GenBank #NP—001068400), Oryza sativa (indica cultivar-group) (GenBank #EAY99617), and Cuphea hookeriana (GenBank #AAC49269). Other TEs include the TesA or TesB from E. coli or YJR019C, YTE1 or YTE2 from yeast or the TE from humans or other mammals.

In some embodiments, at least one TE gene is from a plant, for example overexpressed acyl-ACP thioesterase gene from Ricinus communis, Jatropha curcas, Diploknema butyracea, Cuphea palustris, or Gossypium hirsutum, or an overexpressed hybrid acyl-ACP thioesterase comprising different thioesterase domains operably fused together (see WO2011116279). Preferably, the hybrid thioesterase includes a terminal region of the acyl-ACP thioesterase from Ricinus communis or a 70, 80, 90 or 95% homolog thereto operably coupled to the remaining portion of the thioesterase from another species. In such manner, enzyme specificity can be tailored for the use in question.

In particular, the microorganism can comprise an overexpressed hybrid acyl-ACP thioesterase comprising the amino terminal region of the thioesterase from Ricinus communis operably coupled to the carboxyl region of the thioesterase from another species. Such microorganisms can be combined with each of the other mutations and overexpressions described herein in any combination.

It is also known to change the chain length of the FFAs by changing the TE. Class I acyl-ACP TEs act primarily on 14- and 16-carbon acyl-ACP substrates; 2) Class II acyl-ACP TEs have broad substrate specificities, with major activities toward 8- and 14-carbon acyl-ACP substrates; and 3) Class III acyl-ACP TEs act predominantly on 8-carbon acyl-ACPs.

For example, most thioesterases exhibit the highest specificities in the C16-C18 range, including A. thaliana FatA (18:1Δ9), Madhuca longifolia FatB (16:0, 16:1, 18:0, 18:1), Coriandrum sativum FatA (18:1Δ9), A. thaliana FatB (16:0, 18:1, 18:0, 16:1), Helianthus annuus FatA (18:1, 16:1), and Brassica juncea FatB2 (16:0, 18:0), among numerous others. Medium-chain acyl-ACP thioesterases include Cuphea palustris FatB1 and C. hookeriana FatB2 (8:0, 10:0), C. palustris FatB2 (14:0, 16:0); and Umbellularia californica FatB (12:0, 12:1, 14:0, 14:1). Arecaceae (palm family) and Cuphea accumulate large quantities of fatty acids that are shorter (between 8 and 12 carbon atoms), and several enzymes are also available in bacteria. Exemplary thioesterase families and common names of their members are shown in Table A:

TABLE A
Thioesterase Families and Common Names of their Members

| | Producing | |
|---|---|---|
| Family | organisms | Genes and/or other names of family members |
| TE1 | A, B, E^{a} | Ach1 |
| TE2 | A, B, E | Acot1-Acot6, BAAT thioesterase |
| TE3 | A, B | tesA, acyl-CoA thioesterase I, protease I, lysophospholipase L1 |
| TE4 | B, E | tesB, acyl-CoA thioesterase II, Acot8 |
| TE5 | B | tesC (ybaW), acyl-CoA thioesterase III |
| TE6 | A, B, E | Acot7 (BACH), Acot11 (BFIT, Them1), Acot12 (CACH), YciA |
| TE7 | B, E | Acot9, Acot10 |
| TE8 | A, B, E | Acot13 (Them2) |
| TE9 | B | YbgC |
| TE10 | B | 4HBT-I |
| TE11 | B | 4HBT-II, EntH (YbdB) |
| TE12 | B, E | DNHA-CoA hydrolase |
| TE13 | A, B | paaI, paaD |
| TE14 | B, E | FatA, FatB |
| TE15 | B | Thioesterase CalE7 |
| TE16 | A, B, E | TE domain of FAS (Thioesterase I), TE domain of PKS or NRP (type I |
| | | thioesterase (TE I)) |
| TE17 | B | TE domain of PKS |
| TE18 | B, E | Thioesterase II, type II thioesterase (TE II) |
| TE19 | B | luxD |
| TE20 | E | ppt1, ppt2, palmitoyl-protein thioesterase |
| TE21 | A, B, E | apt1, apt2, acyl-protein thioesterase, phospholipase, carboxylesterase |
| TE22 | A, B, E | S-formylglutathione hydrolase, esterase A, esterase D |
| TE23 | A, B, E | Hydroxyglutathione hydrolase, glyoxalase II |

^{a}A, archaea; B, bacteria E, eukaryota. Most prevalent producers bolded

The TE from Umbellularia californica, which primarily hydrolyzes lauroyl-ACP may be selected as a suitable TE for two reasons. First, it provided FFA titers significantly higher than other acyl-ACP thioesterases, with titers of C12 to C14 species of approximately 180 mg/L. Secondly, the product would be undecane, and the products of in vivo esterification would be lauric acid methyl or ethyl esters, both of which should exhibit desirable properties as diesel fuel replacements or as components in diesel blends.

Our initial cloning experiments proceeded in E. coli for convenience since the needed genes were already available in plasmids suitable for expression in E. coli, and several of the tested strains were already available, but the addition of genes to bacteria is of nearly universal applicability, so it will be possible to use a wide variety of organisms with the selection of suitable vectors for same.

Bacteria from a wide range of species have been successfully modified, and may be the easiest to transform and culture, since the methods were invented in the 70's and are now so commonplace, that even school children perform genetic engineering experiments using bacteria. Such species include e.g., Bacillus, Streptomyces, Azotobacter, Trichoderma, Rhizobium, Pseudomonas, Micrococcus, Nitrobacter, Proteus, Lactobacillus, Pediococcus, Lactococcus, Salmonella, and Streptococcus, or any of the completely sequenced bacterial species. Indeed, hundreds of bacterial genomes have been completely sequenced, and this information greatly simplifies both the generation of vectors encoding the needed genes, as well as the planning of a recombinant engineering protocol. Such species are listed along with links at http://en.wikipedia.org/wiki/List_of_sequenced_bacterial_genomes.

Additionally, yeast are a common species used for microbial manufacturing, and many species can be successfully transformed. In fact, rat TE has already been successfully expressed in yeast Saccharomyces. Other species include but are not limited to Candida, Aspergillus, Arxula adeninivorans, Candida boidinii, Hansenula polymorphs (Pichia angusta), Kluyveromyces lactis, Pichia pastoris, Saccharomyces cerevisiae and Yarrowia lipolytica, to name a few.

It is also possible to genetically modify many species of algae, including e.g., Spirulina, Apergillus, Chlamydomonas, Laminaria japonica, Undaria pinnatifida, Porphyra, Eucheuma, Kappaphycus, Gracilaria, Monostroma, Enteromorpha, Arthrospira, Chlorella, Dunaliella, Aphanizomenon, Isochrysis, Pavlova, Phaeodactylum, Ulkenia, Haematococcus, Chaetoceros, Nannochloropsis, Skeletonema, Thalassiosira, and Laminaria japonica, plus any of the algal species named above. Indeed, the microalga Pavlova lutheri is already being used as a source of economically valuable docosahexaenoic (DHA) and eicosapentaenoic acids (EPA), and Crypthecodinium cohnii is the heterotrophic algal species that is currently used to produce the DHA used in many infant formulas.

Furthermore, a number of databases include vector information and/or a repository of vectors that can be selected for use in these various microbes. See e.g., Addgene.org which provides both a repository and a searchable database allowing vectors to be easily located and obtained from colleagues. See also Plasmid Information Database (PlasmID) and DNASU having over 191,000 plasmids. A collection of cloning vectors of E. coli is also kept at the National Institute of Genetics as a resource for the biological research community. Furthermore, vectors (including particular ORFS therein) are usually available from colleagues.

As used herein, “fatty acids” means any saturated or unsaturated aliphatic acids having the common formulae of CₙH_{2n±x}COOH, wherein x≦n, which contains a single carboxyl group. “Odd chain” fatty acids have an odd number of carbons in the chain (n=even), whereas “even chain” have an even number (n=odd).

As used herein, “reduced activity” is defined herein to be at least a 75% reduction in protein activity, as compared with an appropriate control species. Preferably, at least 80, 85, 90, 95% reduction in activity is attained, and in the most preferred embodiment, the activity is eliminated (100%). Proteins can be inactivated with inhibitors, by mutation, or by suppression of expression or translation, by knock-out, by adding stop codons, by frame shift mutation, and the like. Reduction in activity is indicated by a negative superscript, e.g., FadD⁻

By “knockout” or “null” mutant what is meant is that the mutation produces almost undetectable amounts of protein activity. A gene can be completely (100%) reduced by knockout or removal of part of all of the gene sequence. Use of a frame shift mutation, early stop codon, point mutations of critical residues, or deletions or insertions, and the like, can also completely inactivate (100%) gene product by completely preventing transcription and/or translation of active protein. All knockout mutants herein are signified by Δgene where the gene name is identified in Table A.

As used herein, “overexpression” or “overexpressed” is defined herein to be at least 150% of protein activity as compared with an appropriate control species, or any expression in a species that otherwise lacks the activity. Preferably, the activity is increased 200-500%. Overexpression can be achieved by mutating the protein to produce a more active form or a form that is resistant to inhibition, by removing inhibitors, or adding activators, and the like. Overexpression can also be achieved by removing repressors, adding multiple copies of the gene to the cell, or up-regulating the endogenous gene, and the like. All overexpressed genes or proteins are signified herein by“⁺”.

As used herein, all accession numbers are to GenBank unless indicated otherwise.

Exemplary gene or protein species are provided herein. However, gene and enzyme nomenclature varies widely (esp. in bacteria), thus any protein (or gene encoding same) that catalyzes the same reaction can be substituted for a named protein herein. Further, while exemplary protein sequence accession numbers are provided herein, each is linked to the corresponding DNA sequence, and to related sequences. Further, related sequences can be identified easily by homology search and requisite activities confirmed as by enzyme assay, as is shown in the art.

E. coli gene and protein names (where they have been assigned) can be ascertained through ecoliwiki.net/ and enzymes can be searched through brenda-enzymes.info/.ecoliwiki.net/ in particular provides a list of alternate nomenclature for each enzyme/gene. Many similar databases are available including UNIPROTKB, PROSITE; 5 EC2PDB; ExplorEnz; PRIAM; KEGG Ligand; IUBMB Enzyme Nomenclature; IntEnz; MEDLINE; and MetaCyc, to name a few.

By convention, genes are written in italic, and corresponding proteins in regular font. E.g., fadD is the gene encoding FadD or acyl-CoA synthetase.

Generally speaking, we have used the gene name and protein names interchangeably herein, based on the protein name as provided in ecoliwiki.net. The use of a protein name as an overexpressed protein (e.g., FabH⁺) signifies that protein expression can occur in ways other than by adding a vector encoding same, since the protein can be upregulated in other ways. The use of FadD⁻ signifies that the protein has been downregulated in some way, whereas the use of ΔfadD means that the gene has been directly downregulated to a null mutant.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as instructions for use, buffers, nutritional ingredients and the like.

The following abbreviations are used herein:

| ABBREVIATION | TERM |
|---|---|
| ACC | acetyl-CoA carboxylase and |
| FA | Fatty Acid |
| FabR | DNA-binding transcriptional repressor |
| FabZ | β-hydroxyacyl-acyl carrier protein |
| fadAB | operon encoding a multifunctional enzyme complex containing |
| | thiolase, 3-hydroxyacyl-coenzyme A dehydrogenase, |
| | crotonase , epimerase, and isomerase activities |
| FadD | acyl-CoA synthetase |
| FadE | acyl-CoA dehydrogenase |
| FFA | Free Fatty Acid |
| glpDFK | glycerol transporter (glpF), ATP-dependent glycerol kinase |
| | (glpK) and glycerol-3-phosphate dehydrogenase (glpD) |
| NADK | NAD Kinase |
| PntAB | membrane-bound transhydrogenase |
| TE | Fatty acyl ACP thioesterase |
| UdhA | soluble transhydrogenase |

### BRIEF DESCRIPTION OF THE DRAWINGS

FIG. 1A-B. Global glycerol productions levels in FIG. 1A, broken down by country in FIG. 1B. From Almeida et al., Biodiesel biorefinery: opportunities and challenges for microbial production of fuels and chemicals from glycerol waste, Biotechnology for Biofuels 5:48 (2012).

FIG. 2A-B. (A) Traditional process flow; (B) Proposed new process design with an additional microbial fermentation process (conversion of either glycerin or biomass feedstock to free fatty acids).

FIG. 3. Simplified free fatty acid production pathway from glycerol in engineered E. coli. E. coli is an exemplary species only, and other microbes could be used in the invention, including e.g., Bacillus, Streptomyces, Shigella, yeast, algae, and the like using standard recombinant engineering techniques and the whole genome sequence data available for many species.

FIG. 4A-E. Schematic diagram of the pBAD33 based plasmids used in the proof of concept experiments herein. pBADN (A), pBADU (B), pBADP (C), pBADNU (D) and pBADNP (E). FIG. 4(A) The 879 bp of gene encoded the NAD kinase (nadK) from E. coli was cloned into the vector pBAD33. FIG. 4(B) The 1401 bp of gene encoded the soluble transhydrogenase UdhA (udhA) from E. coli was cloned into the vector pBAD33. FIG. 4(C) The 2932 bp of gene encoded the membrane-bound transhydrogenase PntAB (pntAB) from E. coli was cloned into the vector pBAD33. FIG. 4(D) The 1401 bp of gene encoded the soluble transhydrogenase UdhA (udhA) from E. coli was cloned into the vector pBADN. FIG. 4(E) The 2932 bp of gene encoded the membrane-bound transhydrogenase PntAB (pntAB) from E. coli was cloned into the vector pBADN. In all constructs, RBS were added in front of these genes, which could then be overexpressed. Abbreviations: ara promoter, arabinose induced promoter; araC, regulator gene of arabinoase promoter; CmR, chloramphenicol resistant gene; pACYC184 ori, origin of replication of plasmid pACYC184; rrnBT1,2, transcriptional terminator of rrnB.

FIG. 5A-B Schematic diagram of the pBAD33 based plasmids. pBADglpFK (A), pBADglpFKD (B). FIG. 5(A) The 2377 bp of genes encoded the glycerol transporter (glpF) and ATP-dependent glycerol kinase (glpK) from E. coli was cloned into the vector pBAD33. FIG. 5(B) The 1506 bp of gene encoded the glycerol-3-phospate dehydrogenase (glpD) from E. coli was cloned into the vector pBADglpFK. In all constructs, ribosomal binding sites (RBS) were added in front of these genes, which were then capable of being overexpressed. Abbreviations: ara promoter, arabinose induced promoter; araC, regulator gene of arabinoase promoter; CmR, chloramphenicol resistant gene; pACYC184 ori, origin of replication of plasmid pACYC184; rrnBT1,2, transcriptional terminator of rrnB.

FIG. 6 is literature reports of FFA production by various bacteria. From Engineering Fatty Acid Overproduction in Escherichia coli for Next-Generation Biofuels by Rebecca M. Lennen (Thesis, 2012).

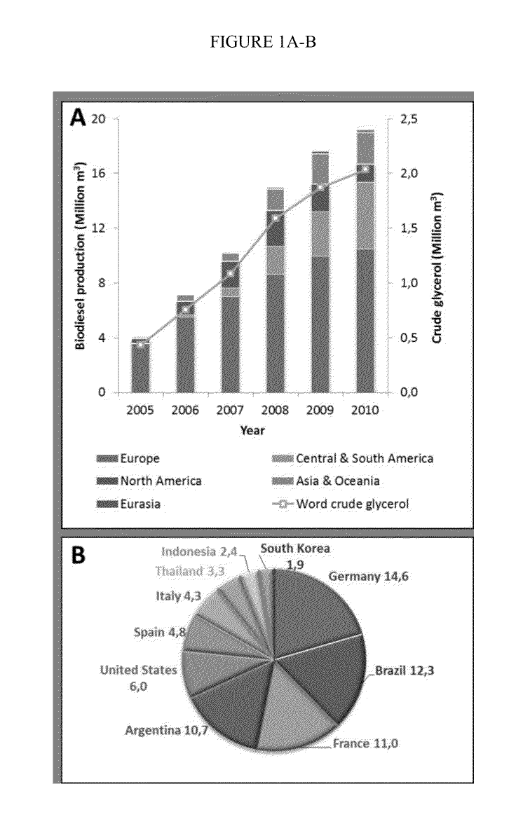

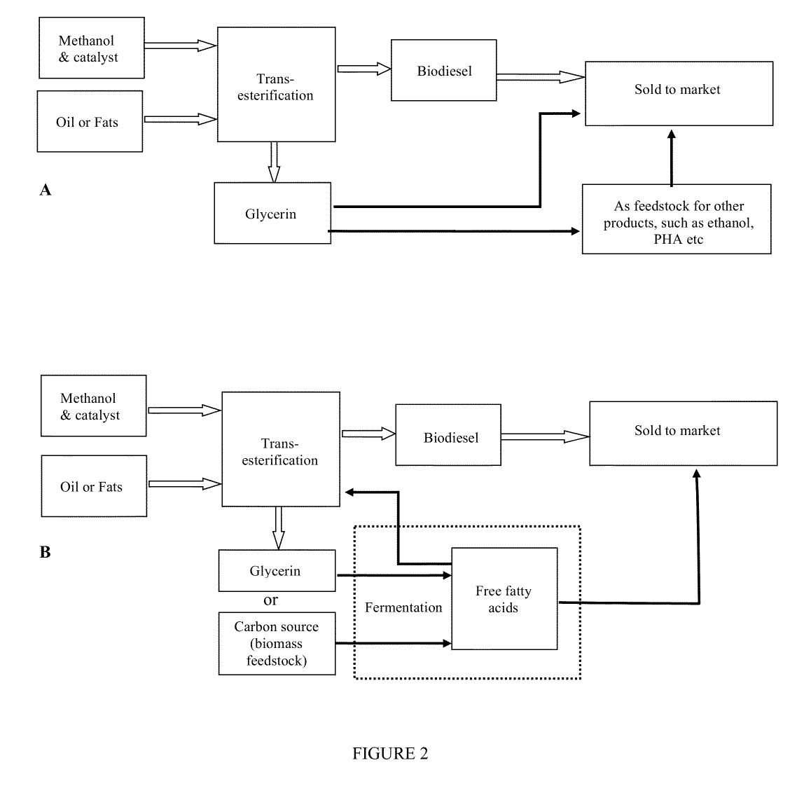

### DETAILED DESCRIPTION

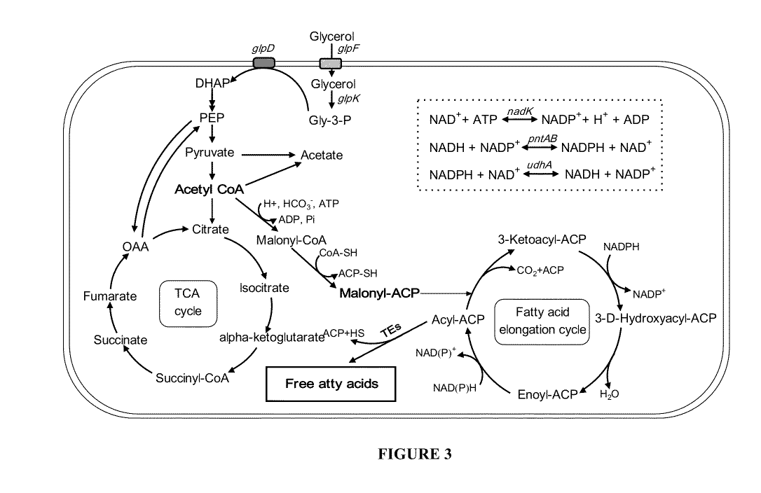

The present invention establishes a method for production of free fatty acid from glycerol by engineered microbes, such as E. coli, yeast, algae, and other microbes. A simplified free fatty acid production pathway from glycerol in engineered E. coli is shown in FIG. 3.

To demonstrate proof of concept, we used a previously engineered E. coli strain ML103 (pXZ18), which can produce high concentration of free fatty acid from glucose, as the starting strain. The ML103 (pXZ18) possessed an acyl-CoA synthetase (fadD⁻) mutation and overexpresses an acyl-ACP thioesterase from Ricinus communis. It is described in more detail in WO2013059218.

It should be noted that ΔfadD is included for convenience only, and is not critical to the invention although it does improve yields. It should also be noted that E. coli typically make negligent amounts of free fatty acids from glycerol, therefore we used the ΔfadD TE⁺ as a baseline bacteria in some experiments because we knew it would make some FFA from our prior work. In other experiments, we used ΔfadD TE⁺ FabZ⁺ as the baseline once we had shown that it was even better than the ΔfadD TE⁺ alone. However, this was only to reduce the number of assays needed, and the results can be compared by multiplying. Furthermore, the optional ΔfadD can also be omitted from the control. Since the fold increase varies with the chosen baseline strain, a better indication of results is provided by the g/L data, which is grams of product per liter of culture medium.

Our work demonstrated that the parent strain ML103 (pXZ18) [ΔfadD TE⁺] can consume glycerol and produce about 1.61 g/L FFAs with the yield of 0.11 g FFAs/g glycerol (Table 2).

The coexpression of alpha-hydroxyacyl-acyl carrier protein dehydratase encoding by fabZ from E. coli with the acyl-ACP thioesterase from R. communis can improve the title and yield to 2.03 g/L and 0.14 g FFAs/g glycerol, respectively (Table 2).

Different mutations of the base strain ML103 were also investigated in our work. The mutation of FabR, the transcriptional repressor of free fatty acid biosynthesis, improved the free fatty acid production. The total concentration of free fatty acid by ML211 (pXZ18Z) increased to 2.33 g/L, about 11.72% higher than that of ML103 (pXZ18Z) (Table 3).

The mutations of acetate formation pathway (poxB and ack-pta), TCA cycle (sucC and pdhR), or PTS system (ptsG) have negative effects on FFAs production using glycerol as the carbon source (Table 3). This is somewhat surprising, since they were expected to improve yield by reducing competition for carbon.

Since the conversion of 1 mole of glycerol can generate 2 mol of NADH and 1 mol of pyruvate, more reducing power (NADH) will be provided when glycerol is used as the carbon source instead of glucose. In addition, NADPH serves as a very important co-factor in the elongation cycle of the FFA biosynthesis. Thus, different combinations of NAD kinase and transhydrogenases can be overexpressed in the free fatty acids producers to further improve yields.

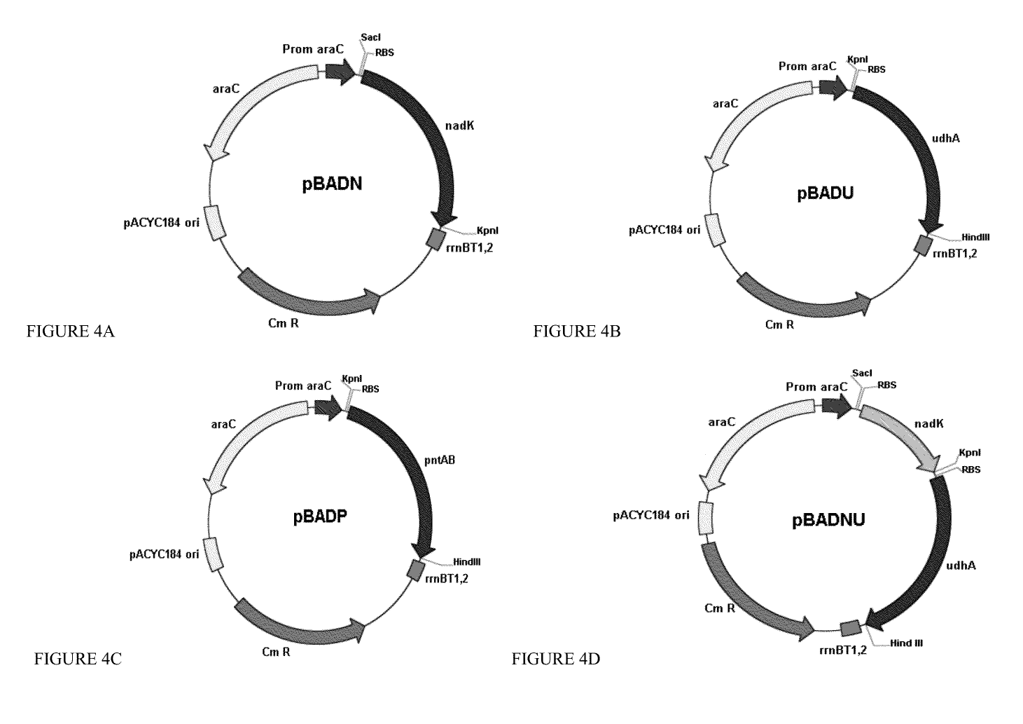

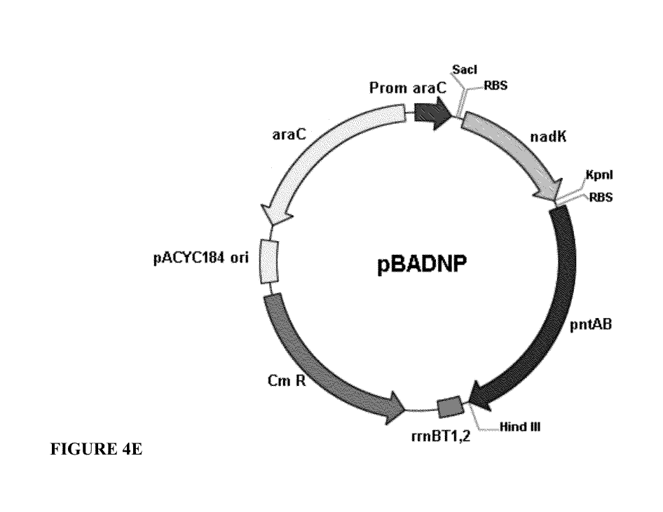

We constructed multiple vectors to overexpress the NAD Kinase—NADK (FIG. 4A), the soluble transhydrogenase—UdhA (FIG. 4B) and the membrane-bound transhydrogenase—PntAB (FIG. 4C). We also constructed the plasmids for co-overexpression of NADK with UdhA (FIG. 4D) and NADK with PntAB (FIG. 4E).

The concentration and yield of total FFAs at 72 hrs in the stains based on ML103 with the overexpression of NADK were improved by 13.9 and 19.68%, respectively. Further improvement of total FFAs and yields of FFAs/g glycerol were achieved when UdhA or PntAB were co-overexpressed with the NADK. The highest concentration and yield of FFAs reached 3.78 g/L and 0.23 g FFAs/g glycerol, respectively, an improvement of 43.58% and 28.20% respectively.

The effect of NADPH manipulation was also examined with the engineered strain MLK211. The concentration and yield of total FFAs in the stains with overexpression of NADK, UdhA or PntAB individually or in combination were all improved (Table 5). The strain MLK211 (pXZ18Z, pBADNP) [ΔfadD TE⁺ NADK⁺, PntAB⁺] with overexpression of NADK and PntAB gave the best improvement in 72 hours. This strain reached 4.82 g/L with a high yield of 0.30 g/g glycerol, respectively. This level of production is extraordinary!

The synergistic effect of strain and cofactor manipulations was examined by re-calculating the improvement with the strain ML103 (pXZ18Z, pBAD33) as the reference using data from Tables 4 and 5. Again, the concentration and yield of total FFAs in the stains with overexpression of NADK, UdhA or PntAB individually or in combination were all improved (Table 6). In this case, the strain MLK211 (pXZ18Z, pBADNP) with overexpression of NADK and PntAB gave the best improvement in both fatty acid and fatty acid/glycerol yield at 72 hours of 83.14% and 64.11%, respectively.

Crude glycerol as a side stream from a commercial biodiesel production plant was added to the LB medium with a final concentration around 15 g/L. The original strain without further modification, ML103(pZX18, pBAD33), was used as the control. The control strain ML103(pXZ18, pBAD33) was capable of producing FFAs using biodiesel crude glycerol as a feedstock, but the total amount of FFAs produced was only 1.98 g/L at 72 h (Table 7). In the case of MLK211(pXZ18Z, pBADNP), the total amount of FFAs increased to 3.53 g/L which was 1.78 times of the control strain (Table 7). The yield of FFAs in MLK211(pXZ18Z, pBADNP) reached 24.13%, which is about 1.85 times that of the control strain (Table 7).

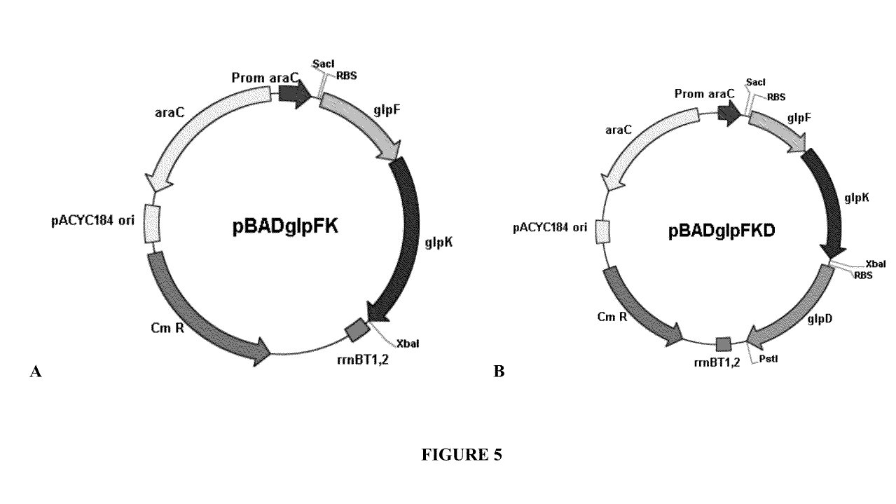

We also manipulated the aerobic glycerol utilization system of E. coli. The whole aerobic glycerol utilization system involves a glycerol transporter (encoded by glpF), an ATP-dependent glycerol kinase (encoded by glpK) and a glycerol-3-phospate dehydrogenase (encoded by glpD). The glpF and glpK locate in one operon in the genome of E. coli and they were cloned into pBAD33 named as pBADglpFK (FIG. 5A). Then the pBADglpFKD was constructed based on pBADglpFK (FIG. 5B), the glpD was cloned into the site followed with glpFK. It is expected that these genetic manipulations alone and together with the redox manipulations (NADK, UdhA, and PntAB) will further improve the productivity of the engineered strains, and work is ongoing to provide these results.

### EXPERIMENTAL METHODS

Aerobic shake flasks experiments were performed at 30° C. with shaking at 250 rpm for 72 h with 1% inoculation in 50 ml LB broth medium supplied with about 15 g/l glycerol and appropriate quantities of kanamycin, chloramphenicol and ampicillin. The concentrations of IPTG were 0.05˜1 mM. Arabinose was added in the medium as an inducer when the strain harbored the pBAD33 based plasmid. The concentrations of arabinose were 0˜50 mM. The initial pH was 7.5. Samples were taken at 24, 48 and 72 h.

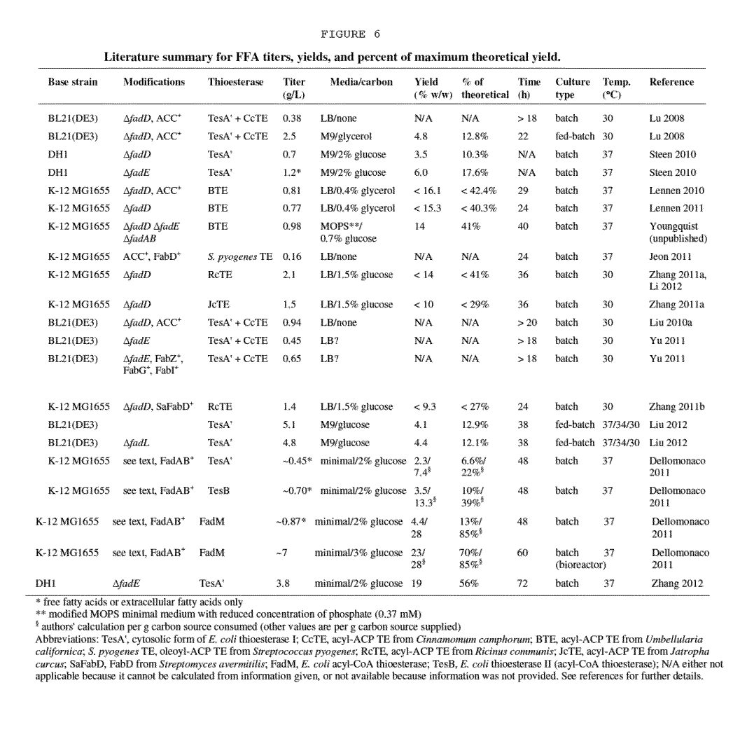

The strains and plasmids used herein are shown in Table 1A and B. Exemplary genes are also given in Table 7 and FIG. 6.

TABLE 1A
Strains used

| Strain | Genotype |
|---|---|
| ML103 | ΔfadD |
| ML151 | ΔfadD, Δack-pta, ΔpoxB, ΔpdhR |
| ML163 | ΔfadD, ΔsucC |
| ML212 | ΔfadD, ΔsucC, ΔfabR |
| ML170 | ΔfadD, ΔphdR |
| ML190 | ΔfadD, ΔptsG |
| ML221 | ΔfadD, ΔptsG, ΔfabR |
| MLK211 | ΔfadD, ΔfabR: Kan |
| ML211 | ΔfadD, ΔfabR |

TABLE 1B
Plasmids used

| | |
|---|---|
| pTrc99a | Cloning vector |
| pBAD33 | Cloning vector |
| pXZ18 (rc_TE⁺) | Overexpressed acyl-ACP thioesterase from R. communis in pTrc99a |
| pXZ18Z (rc_TE⁺, ec_FabZ⁺) | Co-overexpressed acyl-ACP thioesterase from R. communis and β- |
| | hydroxyacyl-acyl carrier protein from E. coli in pTrc99a |
| pBAD33 | Inducible expression vector that is induced by arabinose |
| pBADN (ec_NADK⁺) | Overexpression of NAD kinase from E. coli in pBAD33 |
| pBADU (ec_UdhA⁺) | Overexpression of soluble transhydrogenase from E. coli in pBAD33 |
| | Overexpression of membrane-bound transhydrogenase from E. coli in |
| pBADP (ec_PntAB⁺) | pBAD33 |
| | Co-overexpression of NAD kinase from E. coli and membrane-bound |
| pBADNU (ec_NADK⁺, ec_UdhA⁺) | transhydrogenase from E. coli and soluble transhydrogenase from E. coli |
| | in pBAD33 |
| pBADNP (ec_NADK⁺, ec_ PntAB⁺) | Co-overexpression of NAD kinase from E. coli and membrane-bound |
| | transhydrogenase from E. coli in pBAD33 |
| pBADglpFK | Co-overexpression of glpF and glpK in pBAD33 |
| pBADglpFKD | Co-overexpression of glpF and glpK and glpD in pBAD33 |

### Experiment 1

Effect of fabZ overexpression on free fatty acid production using glycerol as carbon source.

TABLE 2
Total fatty acid production and yield of free fatty acid production at 24, 48, 72 hours

| | Genetic | | Total fatty | | % improvement | % improvement |
|---|---|---|---|---|---|---|
| Strain | type | Time | acids (g/L) | Yield (g/g) | (fatty acids) | (yield) |
| ML103 (pXZ18) | ΔfadD, | 24 h | 1.17 | 0.14 | 0.00 | 0.00 |
| | rc_TE⁺ | 48 h | 1.37 | 0.12 | 0.00 | 0.00 |
| | | 72 h | 1.61 | 0.11 | 0.00 | 0.00 |
| ML103 (pXZ18Z) | ΔfadD, | 24 h | 1.51 | 0.17 | 28.92 | 21.94 |
| | rc_TE⁺, | 48 h | 1.69 | 0.15 | 23.53 | 23.41 |
| | ec_FabZ⁺ | 72 h | 2.03 | 0.14 | 26.23 | 25.89 |

rc_TE⁺: overexpression of acyl-ACP thioesterase from R. communis;
ec_FabZ⁺: overexpression of β-hydroxyacyl-acyl carrier protein from E. coli

### Experiment 2

Effect of different mutation on free fatty acid production using glycerol as carbon source.

TABLE 3
Total fatty acid production and yield of free fatty acid production at 24, 48, 72 hours

| | | | Total fatty | | % improvement | % improvement |
|---|---|---|---|---|---|---|
| Strain | Genetic type | Time | acids (g/L) | Yield (g/g) | (fatty acids) | (yield) |
| ML103 (pXZ18Z) | ΔfadD, rc_TE⁺, | 24 h | 1.51 | 0.15 | 0.00 | 0.00 |
| | ec_FabZ⁺ | 48 h | 1.82 | 0.15 | 0.00 | 0.00 |
| | | 72 h | 2.09 | 0.13 | 0.00 | 0.00 |
| ML151 (pXZ18Z) | ΔfadD, Δack-pta, | 24 h | 0.25 | 0.04 | −83.18 | −73.48 |
| | ΔpoxB, ΔpdhR, | 48 h | 0.37 | 0.04 | −79.73 | −73.71 |
| | rc_TE⁺, ec_FabZ⁺ | 72 h | 0.35 | 0.04 | −83.02 | −73.03 |
| ML163 (pXZ18Z) | ΔfadD, ΔsucC, rc_TE⁺, | 24 h | 0.53 | 0.12 | −65.15 | −18.87 |
| | ec_FabZ⁺ | 48 h | 0.59 | 0.13 | −67.28 | −17.72 |
| | | 72 h | 0.59 | 0.04 | −71.94 | −69.64 |
| ML212 (pXZ18Z) | ΔfadD, ΔsucC, ΔfabR, | 24 h | 0.89 | 0.14 | −40.93 | −7.44 |
| | rc_TE⁺, ec_FabZ⁺ | 48 h | 1.04 | 0.15 | −42.80 | −3.69 |
| | | 72 h | 1.13 | 0.08 | −46.03 | −43.36 |
| ML170 (pXZ18Z) | ΔfadD, ΔphdR, | 24 h | 1.16 | 0.11 | −23.76 | −30.21 |
| | rc_TE⁺, ec_FabZ⁺ | 48 h | 1.38 | 0.11 | −24.31 | −25.91 |
| | | 72 h | 1.60 | 0.11 | −23.26 | −20.66 |
| ML190 (pXZ18Z) | ΔfadD, ΔptsG, rc_TE⁺, | 24 h | 1.43 | 0.15 | −5.88 | −4.08 |
| | ec_FabZ⁺ | 48 h | 1.77 | 0.15 | −2.59 | 1.35 |
| | | 72 h | 2.01 | 0.13 | −3.61 | −3.40 |
| ML221 (pXZ18Z) | ΔfadD, ΔptsG, ΔfabR, | 24 h | 1.35 | 0.13 | −10.56 | −14.21 |
| | rc_TE⁺, ec_FabZ⁺ | 48 h | 1.81 | 0.15 | −0.33 | −3.55 |
| | | 72 h | 2.19 | 0.14 | 5.05 | 1.84 |
| ML211 (pXZ18Z) | ΔfadD, ΔfabR, rc_TE⁺, | 24 h | 1.64 | 0.15 | 7.99 | −0.93 |
| | ec_FabZ⁺ | 48 h | 2.00 | 0.16 | 9.88 | 4.41 |
| | | 72 h | 2.33 | 0.14 | 11.72 | 7.84 |

rc_TE⁺: overexpression of acyl-ACP thioesterase from R. communis;
ec_FabZ⁺: overexpression of β-hydroxyacyl-acyl carrier protein from E. coli

### Experiment 3

Effect of nadK, udhA and pntAB overexpression on free fatty acid production using glycerol as carbon source.

TABLE 4
Total fatty acid production and yield of free fatty acid production at 24, 48, 72 hours

| | Genetic | | Total fatty | Yield | % improvement | % improvement |
|---|---|---|---|---|---|---|
| Stain | type | Time | acids (g/L) | (g/g) | (fatty acids) | (yield) |
| ML103 (pXZ18Z, | ΔfadD, | 24 h | 0.86 | 0.28 | 0 | 0 |
| pBAD33) | rc_TE⁺, | 48 h | 1.51 | 0.20 | 0 | 0 |
| | ec_FabZ⁺ | 72 h | 2.63 | 0.18 | 0 | 0 |
| ML103 (pXZ18Z, | ΔfadD, | 24 h | 1.06 | 0.33 | 23.80 | 18.23 |
| pBADN) | rc_TE⁺, | 48 h | 1.90 | 0.18 | 25.97 | −10.55 |
| | ec_FabZ⁺, | 72 h | 3.00 | 0.21 | 13.90 | 19.68 |
| | ec_NADK⁺ | | | | | |
| ML103 (pXZ18Z, | ΔfadD, | 24 h | 1.01 | 0.25 | 17.34 | −8.78 |
| pBADU) | rc_TE⁺, | 48 h | 2.17 | 0.22 | 43.62 | 12.57 |
| | ec_FabZ⁺, | 72 h | 2.91 | 0.19 | 10.63 | 8.23 |
| | ec_UdhA⁺ | | | | | |
| ML103 (pXZ18Z, | ΔfadD, | 24 h | 1.17 | 0.27 | 36.29 | −1.54 |
| pBADNU) | rc_TE⁺, | 48 h | 2.04 | 0.26 | 34.86 | 31.77 |
| | ec_FabZ⁺, | 72 h | 3.42 | 0.21 | 29.96 | 18.45 |
| | ec_NADK⁺, | | | | | |
| | ec_UdhA⁺ | | | | | |
| ML103 (pXZ18Z, | ΔfadD, | 24 h | 1.39 | 0.22 | 62.28 | −18.98 |
| pBADNP) | rc_TE⁺, | 48 h | 2.44 | 0.23 | 61.59 | 14.79 |
| | ec_FabZ⁺, | 72 h | 3.78 | 0.23 | 43.58 | 28.20 |
| | ec_NADK⁺, | | | | | |
| | ec_PntAB⁺ | | | | | |

rc_TE⁺: overexpression of acyl-ACP thioesterase from R. communis;
ec_FabZ⁺: overexpression of β-hydroxyacyl-acyl carrier protein from E. coli
ec_NADK⁺: overexpression of NAD kinase from E. coli
ec_UdhA⁺: overexpression of soluble transhydrogenase from E. coli
ec_PntAB⁺: overexpression of membrane-bound transhydrogenase from E. coli

### Experiment 4

Effect of nadK, udhA and pntAB overexpression on free fatty acid production using glycerol as carbon source using MLK211 engineered strain.

TABLE 5
Total fatty acid production and yield of free fatty acid production at 24, 48, 72 hours.

| | Genetic | | Total fatty | Yield | % improvement | % improvement |
|---|---|---|---|---|---|---|
| Stain | type | Time | acids (g/L) | (g/g) | (fatty acids) | (yield) |
| MLK211(pXZ18Z, | ΔfadD, | 24 h | 1.86 | 0.25 | 0 | 0 |
| pBAD33) | ΔfabR, | 48 h | 2.68 | 0.24 | 0 | 0 |
| | rc_TE⁺, | 72 h | 3.56 | 0.22 | 0 | 0 |
| | ec_FabZ⁺ | | | | | |
| MLK211(pXZ18Z, | ΔfadD, | 24 h | 2.51 | 0.31 | 34.72 | 24.70 |
| pBADN) | ΔfabR, | 48 h | 3.29 | 0.29 | 22.79 | 17.89 |
| | rc_TE⁺, | 72 h | 4.48 | 0.28 | 25.97 | 25.40 |
| | ec_FabZ⁺, | | | | | |
| | ec_NADK⁺ | | | | | |
| MLK211 (pXZ18Z, | ΔfadD, | 24 h | 2.41 | 0.30 | 29.32 | 18.11 |
| pBADU) | ΔfabR, | 48 h | 3.09 | 0.27 | 15.39 | 9.64 |
| | rc_TE⁺, | 72 h | 4.19 | 0.26 | 17.88 | 17.90 |
| | ec_FabZ⁺, | | | | | |
| | ec_UdhA⁺ | | | | | |
| MLK211 (pXZ18Z, | ΔfadD, | 24 h | 2.36 | 0.27 | 26.78 | 7.46 |
| pBADP) | ΔfabR, | 48 h | 3.22 | 0.28 | 20.30 | 16.16 |
| | rc_TE⁺, | 72 h | 4.15 | 0.25 | 16.54 | 14.08 |
| | ec_FabZ⁺, | | | | | |
| | ec_PntAB⁺ | | | | | |
| MLK211 (pXZ18Z, | ΔfadD, | 24 h | 2.18 | 0.26 | 16.94 | 2.54 |
| pBADNU) | ΔfabR, | 48 h | 3.20 | 0.20 | 19.44 | 26.95 |
| | rc_TE⁺, | 72 h | 4.52 | 0.28 | 27.17 | 24.57 |
| | ec_FabZ+, | | | | | |
| | ec_NADK⁺, | | | | | |
| | ec_UdhA⁺ | | | | | |
| MLK211 (pXZ18Z, | ΔfadD, | 24 h | 2.16 | 0.27 | 16.21 | 8.40 |
| pBADNP) | ΔfabR, | 48 h | 3.03 | 0.25 | 13.14 | 2.41 |
| | rc_TE⁺, | 72 h | 4.82 | 0.30 | 35.39 | 33.55 |
| | ec_FabZ⁺, | | | | | |
| | ec_NADK⁺, | | | | | |
| | ec_PntAB⁺ | | | | | |

rc_TE⁺: overexpression of acyl-ACP thioesterase from R. communis;
ec_FabZ⁺: overexpression of β-hydroxyacyl-acyl carrier protein from E. coli
ec_NADK⁺: overexpression of NAD kinase from E. coli
ec_UdhA⁺: overexpression of soluble transhydrogenase from E. coli
ec_PntAB⁺: overexpression of membrane-bound transhydrogenase from E. coli

### Experiment 5

Synergistic effect of combining host strain manipulation with nadK, udhA and pntAB overexpression on free fatty acid production using glycerol as carbon source using MLK211 engineered strain (data taken from Table 4 and Table 5).

TABLE 6
Total fatty acid production and yield of free fatty acid production at 24, 48, 72 hours

| | Genetic | | Total fatty | Yield | % improvement | % improvement |
|---|---|---|---|---|---|---|
| Stain | type | Time | acids (g/L) | (g/g) | (fatty acids) | (yield) |
| ML103 (pXZ18Z, | ΔfadD, | 24 h | 0.86 | 0.28 | 0 | 0 |
| pBAD33) | rc_TE⁺, | 48 h | 1.51 | 0.20 | 0 | 0 |
| | ec_FabZ⁺ | 72 h | 2.63 | 0.18 | 0 | 0 |
| MLK211(pXZ18Z, | ΔfadD, | 24 h | 2.51 | 0.31 | 191.39 | 11.31 |
| pBADN) | ΔfabR, | 48 h | 3.29 | 0.29 | 117.90 | 42.51 |
| | rc_TE⁺, | 72 h | 4.48 | 0.28 | 70.41 | 54.10 |
| | ec_FabZ⁺, | | | | | |
| | ec_NADK⁺ | | | | | |
| MLK211 (pXZ18Z, | ΔfadD, | 24 h | 2.41 | 0.30 | 179.73 | 5.42 |
| pBADU) | ΔfabR, | 48 h | 3.09 | 0.27 | 104.76 | 32.54 |
| | rc_TE⁺, | 72 h | 4.19 | 0.26 | 59.45 | 44.88 |
| | ec_FabZ⁺, | | | | | |
| | ec_UdhA⁺ | | | | | |
| MLK211 (pXZ18Z, | ΔfadD, | 24 h | 2.36 | 0.27 | 174.22 | −4.08 |
| pBADP) | ΔfabR, | 48 h | 3.22 | 0.28 | 113.48 | 40.42 |
| | rc_TE⁺, | 72 h | 4.15 | 0.25 | 57.65 | 40.18 |
| | ec_FabZ⁺, | | | | | |
| | ec_PntAB⁺ | | | | | |
| MLK211 (pXZ18Z, | ΔfadD, | 24 h | 2.18 | 0.26 | 152.94 | −8.47 |
| pBADNU) | ΔfabR, | 48 h | 3.20 | 0.20 | 111.99 | 53.46 |
| | rc_TE⁺, | 72 h | 4.52 | 0.28 | 72.02 | 53.08 |
| | ec_FabZ+, | | | | | |
| | ec_NADK⁺, | | | | | |
| | ec_UdhA⁺ | | | | | |
| MLK211 (pXZ18Z, | ΔfadD, | 24 h | 2.16 | 0.27 | 151.36 | −3.24 |
| pBADNP) | ΔfabR, | 48 h | 3.03 | 0.25 | 100.78 | 23.79 |
| | rc_TE⁺, | 72 h | 4.82 | 0.30 | 83.14 | 64.11 |
| | ec_FabZ⁺, | | | | | |
| | ec_NADK⁺, | | | | | |
| | ec_PntAB⁺ | | | | | |

rc_TE⁺: overexpression of acyl-ACP thioesterase from R. communis;
ec_FabZ⁺: overexpression of β-hydroxyacyl-acyl carrier protein from E. coli
ec_NADK⁺: overexpression of NAD kinase from E. coli
ec_UdhA⁺: overexpression of soluble transhydrogenase from E. coli
ec_PntAB⁺: overexpression of membrane-bound transhydrogenase from E. coli

### Example 6

Free fatty acid production using a commercial biodiesel based crude glycerol as the carbon source.

TABLE 7
Total fatty acid production and yield of free fatty acid production at 24, 48, 72 hours

| | | | Total fatty | | % improvement | % improvement |
|---|---|---|---|---|---|---|
| Strain | Genetic type | Time | acids (g/L) | Yield (g/g) | (fatty acids) | (yield) |
| ML103 (pXZ18, | ΔfadD, | 24 h | 0.49 | 21.57 | — | — |
| pBAD33) | rc_TE⁺ | 48 h | 1.37 | 13.66 | — | — |
| | | 72 h | 1.98 | 13.02 | — | — |
| MLK211 | ΔfadD, | 24 h | 0.73 | 28.11 | 50.83 | 30.30 |
| (pXZ18Z, pBADNP) | ΔfabR, | 48 h | 2.04 | 24.84 | 49.15 | 81.82 |
| | rc_TE⁺, | 72 h | 3.53 | 24.13 | 78.47 | 85.24 |
| | ec_FabZ⁺, | | | | | |
| | ec_NADK⁺, | | | | | |
| | ec_PntAB⁺ | | | | | |

rc_TE⁺: overexpression of acyl-ACP thioesterase from R. communis;
ec_FabZ⁺: overexpression of β-hydroxyacyl-acyl carrier proteinfrom E. coli;
ec_NADK⁺: overexpression of NAD kinase from E. coli;
ec_PntAB⁺: overexpression of membrane-bound transhydrogenase from E. coli

TABLE 8
Gene name and related information

| | | GenBank | |
|---|---|---|---|
| | | Accession | |
| Strain | Gene | or Gene ID | Protein_ID |
| Escherichia coli | fabZ | 944888 | AAC73291.1 |
| Escherichia coli | nadK | 947092 | AAC75664.1 |
| Escherichia coli | udhA | 948461 | AAC76944.2 |
| Escherichia coli | pntA | 946628 | AAC74675.1 |
| Escherichia coli | pntB | 946144 | AAC74674.1 |
| Escherichia coli | glpF | 948422 | AAC76909.1 |
| Escherichia coli | glpD | 12932271 | BAE77866.1 |
| Escherichia coli | glpK | 948423 | AAC76908.1 |

Other bacteria that can be used in the invention are shown in FIG. 6. These bacteria are all readily available (indeed several were made by our group) and have been tested for FFA production levels.

It is also possible to put the same overexpressed TE gene(s) into yeast and/or algae, although the ORF should be moved to expression vectors that are optimized for yeast and/or algae. Experiments may be planned to move the TE from Ricinus or Cuphea, both of which are good TE's, into yeast and/or algae. In either species, it may be preferred to re-synthesize the TE gene, using codons optimized for use in that species, especially in the algae.

For example, the yeast expression vector pKLAC1 directs high-level expression of a recombinant protein from the yeast Kluyveromyces lactis and contains the strong K. lactis P_{LAC4-PBI} promoter, DNA encoding the K. lactis á-mating factor (á-MF) secretion domain (for secreted expression), a multiple cloning site (MCS), the K. lactis LAC4 transcription terminator (TT), and a fungal acetamidase selectable marker gene (amdS) expressed from the yeast ADH2 promoter (P_{ADH2}). An E. coli replication origin (ori) and ampicillin resistance gene (Ap^{R}) are also present for propagation of pKLAC1 in E. coli.

The pKLAC1 vector is exemplary only. IP-Free© yeast expression vectors for high levels of protein expression with a choice of promoters are also available for Pichia pastoris or Saccharomyces cerevisiae at DNA 2.0, pTEF1/Zeo and pTEF1/Bsd are available from Invitrogen, YeastXceed™ Technology is available from Creative BiolAbs, and hundreds more yeast expression vectors are available in ADDGENE.

Yeast can grow on glycerol, so no further manipulation is required, although as with bacteria, additional changes can be made to optimize FFA production. For example, glycerol permease, glycerol kinase and glycerophosphate oxidase can be overexpressed, and pathways competing for carbon resources can be reduced. Yeast are also available that can grow on pure glycerol, and these strains (Yarrowia lipolytica (DiSVA C 12.1), Metschnikowia sp. (DiSVA 50), Debaryomyces sp. (DiSVA 45/9), and Rhodotorula mucilaginosa (DiSVA C 7.1)) may be particularly good hosts for this work. Taccari (2012).

Historically, the green algae Chlamydomonas reinhardtii has been the focus of most molecular and genetic physiological research. Therefore, most of the tools for the expression of transgenes and gene knockdown have been developed for and are specific for this species. However, tools are now also being rapidly developed for diatoms and other algae that are of greater interest for industrial applications. The stability of expression can be improved through proper codon usage, the use of strong endogenous promoters, and inclusion of species-specific 5′, 3′, and intron sequences. The efficiency of transformation is strongly species dependent, and the method of transformation has to be carefully selected and optimized for each microalga.

GeneArt® kits are commercially available for genetic modification and expression for the photosynthetic microalgae Chlamydomonas reinhardtii 137c and Synechococcus elongatus PPC 7942. The most recent kits are optimized for high levels of protein expression with dual protein tags for detection and purification as well as selection against gene silencing often seen in Chlamydomonas. As with yeast, algae can utilize glycerol as a carbon source, indeed, considerable research is already underway using algae to produce oils on glycerol substrates. Thus, existing algae can easily be used in the disclosed method. As with bacteria, the algae can be optimized to increase FFA production, such as starch-deficient strains of C. reinhardtii, the sta6 and sta7 mutants. Radakovits (2010).

A variety of transformation methods have been used to transfer DNA into microalgal cells, including agitation in the presence of glass beads or silicon carbide whiskers, electroporation, biolistic microparticle bombardment, and Agrobacterium tumefaciens-mediated gene transfer. In fact, successful genetic transformation has been reported for the green (Chlorophyta), red (Rhodophyta), and brown (Phaeophyta) algae; diatoms; euglenids; and dinoflagellates.

The following are incorporated by reference herein in their entireties for all purposes:
WO2013059218—Bacteria and method for synthesizing fatty acids
WO2011116279—Bacteria and method for synthesizing fatty acids
61/740,959, filed Dec. 21, 2012 and Ser. No. 14/104,628 filed Dec. 12, 2013 MICROBIAL ODD CHAIN FATTY ACIDS
61/809,759, filed Apr. 8, 2013 IMPROVING FATTY ACID BIOPRODUCTION
Janβen H. J. & Steinbüchel A., Fatty acid synthesis in Escherichia coli and its applications towards the production of fatty acid based biofuels, Biotechnology for Biofuels 7:7 (2014).
Li, M., et al., Effect of acetate formation pathway and long chain fatty acid CoA-ligase on the free fatty acid production in E. coli expressing acyl-ACP thioesterase from Ricinus communis. Metab. Eng. 14:380-387 (2012).
Radakovits R., et al., Genetic Engineering of Algae for Enhanced Biofuel Production, Eukaryotic Cell 9(4): 486-501 (2010).
Taccari M, et al., Screening of yeasts for growth on crude glycerol and optimization of biomass production Bioresour Technol. 2012 April; 110:488-95 (2012).
Zhang, X. et al, Efficient free fatty acid production in Escherichia coli using plant acyl-ACP thioesterases, Metabolic Engineering 13(6): 713-22 (2011).
Zhang, X., Improving fatty acid production in Escherichia coli through the overexpression of malonyl CoA-acyl carrier protein transacylase. Biotechnol. Prog. 28:60-65 (2011).
Cantu, D. C. et al., Thioesterases: A new perspective based on their primary and tertiary structures, Protein Sci. July 2010; 19(7): 1281-1295 (2010).
Engineering Fatty Acid Overproduction in Escherichia coli for Next-Generation Biofuels by Rebecca M. Lennen (thesis, http://depot.library.wise.edu/repository/fedora/1711.dl:TILJIP5I5DABR82/datastreams/REF/content).

1. A method of biodiesel production comprising:
a) converting a biologically produced triglycerides to free fatty acids and glycerol;
b) converting said free fatty acids to biodiesel by esterification of said free fatty acids;
c) separating said glycerol from said free fatty acids or biodiesel;
d) converting the separated glycerol to free fatty acids in a reactor with a recombinant microbe that has been engineered to comprise an overexpressed fatty acyl ACP thioesterase (TE) and that converts glycerol to free fatty acids;
e) separating said free fatty acids from said recombinant microbe; and,
f) converting the separated free fatty acids to biodiesel.

2. The method of claim 1, wherein all steps are performed at the same plant.

3. The method of claim 1, wherein the free fatty acids from step e are routed to a reactor that performs step b.

4. The method of claim 1, wherein said recombinant microbe comprises one of the following genotypes:
TE⁺ wherein TE is an acyl-ACP thioesterase
ΔfadD, TE⁺ wherein FadD is acyl-CoA synthetase
ΔfadD, TE⁺, FabZ⁺ wherein FabZ is β-hydroxyacyl-acyl carrier protein
TE⁺, NADK⁺ wherein NADK is NAD kinase
TE⁺, UdhA⁺ wherein UdhA is soluble transhydrogenase
TE⁺, PntAB⁺ wherein PntAB is membrane-bound transhydrogenase
TE⁺, NADK⁺, UdhA⁺
TE⁺, NADK⁺, PntAB⁺
TE⁺, FabZ⁺, NADK⁺
TE⁺, FabZ⁺, UdhA⁺
TE⁺, FabZ⁺, PntAB⁺
TE⁺, FabZ⁺, NADK⁺, UdhA⁺
TE⁺, FabZ⁺, NADK⁺, PntAB⁺
ΔfadD, TE⁺, NADK⁺
ΔfadD, TE⁺, UdhA⁺
ΔfadD, TE⁺, PntAB⁺
ΔfadD, TE⁺, NADK⁺, UdhA⁺
ΔfadD, TE⁺, NADK⁺, PntAB⁺
ΔfadD, TE⁺, FabZ⁺, NADK⁺,
ΔfadD, TE⁺, FabZ⁺, UdhA⁺
ΔfadD, TE⁺, FabZ⁺, PntAB⁺
ΔfadD, TE⁺, FabZ⁺, NADK⁺, UdhA⁺
ΔfadD, TE⁺, FabZ⁺, NADK⁺, PntAB⁺
ΔfadD, ΔfabR, TE⁺, FabZ⁺
ΔfadD, ΔfabR, TE⁺, FabZ⁺, NADK⁺
ΔfadD, ΔfabR, TE⁺, FabZ⁺, UdhA⁺
ΔfadD, ΔfabR, TE⁺, FabZ⁺, PntAB⁺
ΔfadD, ΔfabR, TE⁺, FabZ⁺, NADK⁺, UdhA⁺
ΔfadD, ΔfabR, TE⁺, FabZ⁺, NADK⁺, PntAB⁺
ΔfadD, ΔfabR, TE⁺, FabZ⁺, NADK⁺, glpDFK⁺ wherein glpDFK is the glycerol
transporter (glpF), ATP-dependent glycerol kinase (glpK) and glycerol-3-phosphate
dehydrogenase (glpD)
TE⁺, glpDFK⁺
TE⁺, ACC⁺ wherein ACC is acetyl-CoA carboxylase
TE⁺, accABCD⁺ wherein AccABCD is the operon for the 4 ACC subunits
ΔfadD, ΔfadE, ΔfadAB, TE⁺ wherein fadE is the gene encoding acyl-CoA dehydrogenase and fadAB
is the gene encoding a multifunctional enzyme complex containing thiolase, 3-hydroxyacyl-coenzyme
A dehydrogenase, crotonase, epimerase, and isomerase activities
glpDFK⁺ added to any genotype herein

5. The method of claim 2, wherein said TE is from Ricinus communis.

6. The method of claim 1, wherein said recombinant microbe makes at least 0.25 g/L of free fatty acid.

7. The method of claim 1, wherein said recombinant microbe makes at least 0.5 g/L of free fatty acid.

8. The method of claim 1, wherein said recombinant microbe makes at least 0.75 g/L of free fatty acid.

9. The method of claim 1, wherein said recombinant microbe makes at least 1 g/L of free fatty acid.

10. The method of claim 1, wherein said recombinant microbe makes at least 2 g/L of free fatty acid.

11. The method of claim 1, wherein said recombinant microbe makes at least 3 g/L of free fatty acid.

12. The method of claim 1, wherein said recombinant microbe makes at least 4 g/L of free fatty acid.

13. A microbe comprising:
TE⁺, NADK⁺
TE⁺, UdhA⁺
TE⁺, PntAB⁺
TE⁺, NADK⁺, UdhA⁺
TE⁺, NADK⁺, PntAB⁺
TE⁺, FabZ⁺, NADK⁺
TE⁺, FabZ⁺, UdhA⁺
TE⁺, FabZ⁺, PntAB⁺
TE⁺, FabZ⁺, NADK⁺, UdhA⁺
TE⁺, FabZ⁺, NADK⁺, PntAB⁺
ΔfadD, TE⁺, NADK⁺
ΔfadD, TE⁺, UdhA⁺
ΔfadD, TE⁺, PntAB⁺
ΔfadD, TE⁺, NADK⁺, UdhA⁺
ΔfadD, TE⁺, NADK⁺, PntAB⁺
ΔfadD, TE⁺, FabZ⁺, NADK⁺
ΔfadD, TE⁺, FabZ⁺, UdhA⁺
ΔfadD, TE⁺, FabZ⁺, PntAB⁺
ΔfadD, TE⁺, FabZ⁺, NADK⁺, UdhA⁺
ΔfadD, TE⁺, FabZ⁺, NADK⁺, PntAB⁺
ΔfadD, ΔfabR, TE⁺, FabZ⁺
ΔfadD, ΔfabR, TE⁺, FabZ⁺, NADK⁺
ΔfadD, ΔfabR, TE⁺, FabZ⁺, UdhA⁺
ΔfadD, ΔfabR, TE⁺, FabZ⁺, PntAB⁺
ΔfadD, ΔfabR, TE⁺, FabZ+, NADK⁺, UdhA⁺
ΔfadD, ΔfabR, TE⁺, FabZ⁺, NADK⁺, PntAB⁺
ΔfadD, ΔfabR, TE⁺, FabZ⁺, NADK⁺, glpDFK⁺
TE⁺, ACC⁺
TE⁺, accABCD⁺
ΔfadD, ΔfadE, ΔfadAB, TE⁺
glpDFK⁺ added to any genotype herein

14. A method of biodiesel production comprising:
a) converting a fat or oil to biodiesel and glycerol;
b) separating said biodiesel from said glycerol;
c) converting the separated glycerol to fatty acids in a reactor containing growth medium and a bacteria comprising an overexpressed TE⁺ gene;
d) separating said fatty acids from said bacteria or said growth medium or both; and
e) converting the separated fatty acids to biodiesel.

15. The method of claim 14, wherein said recombinant bacteria has one of the following genotypes:
TE⁺
ΔfadD, TE⁺
ΔfadD, TE⁺, FabZ⁺
TE⁺, NADK⁺
TE⁺, UdhA⁺
TE⁺, PntAB⁺
TE⁺, NADK⁺, UdhA⁺
TE⁺, NADK⁺, PntAB⁺
TE⁺, FabZ⁺, NADK⁺
TE⁺, FabZ⁺, UdhA⁺
TE⁺, FabZ⁺, PntAB⁺
TE⁺, FabZ⁺, NADK⁺, UdhA⁺
TE⁺, FabZ⁺, NADK⁺, PntAB⁺
ΔfadD, TE⁺, NADK⁺
ΔfadD, TE⁺, UdhA⁺
ΔfadD, TE⁺, PntAB⁺
ΔfadD, TE⁺, NADK⁺, UdhA⁺
ΔfadD, TE⁺, NADK⁺, PntAB⁺
ΔfadD, TE⁺, FabZ⁺, NADK⁺
ΔfadD, TE⁺, FabZ⁺, UdhA⁺
ΔfadD, TE⁺, FabZ⁺, PntAB⁺
ΔfadD, TE⁺, FabZ⁺, NADK⁺, UdhA⁺
ΔfadD, TE⁺, FabZ⁺, NADK⁺, PntAB⁺
ΔfadD, ΔfabR, TE⁺, FabZ⁺
ΔfadD, ΔfabR, TE⁺, FabZ⁺, NADK⁺
ΔfadD, ΔfabR, TE⁺, FabZ⁺, UdhA⁺
ΔfadD, ΔfabR, TE⁺, FabZ⁺, PntAB⁺
ΔfadD, ΔfabR, TE⁺, FabZ+, NADK⁺, UdhA⁺
ΔfadD, ΔfabR, TE⁺, FabZ⁺, NADK⁺, PntAB⁺
ΔfadD, ΔfabR, TE⁺, FabZ⁺, NADK⁺, glpDFK⁺
TE⁺, glpDFK⁺
TE⁺, ACC⁺
TE⁺, accABCD⁺
ΔfadD, ΔfadE, ΔfadAB, TE⁺
glpDFK⁺ with any genotype herein

16. The method of claim 15, wherein said bacteria are grown under aerobic conditions followed by microaerobic conditions.

17. The method of claim 15, wherein said growth medium comprises 0.1-1% acetic acid.

18. The method of claim 15, wherein said free fatty acids are secreted by said bacteria and harvested from said growth medium.

19. A method of biodiesel production comprising:
a) growing a recombinant bacteria comprising an overexpressed plant acyl-ACP thioesterase in a reactor containing a growth medium comprising a glycerol produced from the synthesis of biodiesel, wherein said bacteria produces free fatty acids from said glycerol;
b) separating said free fatty acids from said bacteria or said growth medium or both; and,
c) esterifying said free fatty acids to make additional biodiesel.

20. The method of claim 19, wherein said reactor is operated under aerobic conditions followed by microaerobic conditions.

21. The method of claim 19, wherein said growth medium comprises 0.1-1% acetic acid.

22. The method of claim 19, wherein said glycerol is both produced and converted to free fatty acid in the same facility.
